(19) United States
(12) Patent Application Publication
(10) Pub. No.: US 2012/0095401 A1
(43) Pub. Date: Apr. 19, 2012

(54) LIQUID SUPPLY PUMP AND MEDICAL INSTRUMENT

(75) Inventors: Kazuaki Uchida, Matsumoto-shi (JP); Takeshi Seto, Chafu-shi (JP); Kazuo Kawasumi, Chino-shi (JP)

(73) Assignee: SEIKO EPSON CORPORATION, Tokyo (JP)

(21) Appl. No.: 13/271,068

(22) Filed: Oct. 11, 2011

(30) Foreign Application Priority Data
Oct. 15, 2010 (JP) 2010-232255

(51) Int. Cl. A61M 1/00; F04B 17/00

(52) U.S. Cl. 604/151; 417/321

(57) ABSTRACT

A liquid supply pump includes: three chambers of a first liquid chamber, a second liquid chamber, and a third liquid chamber disposed in series and produced by dividing a space between an inlet channel into which liquid is supplied and an outlet channel from which liquid is delivered; a first movable partition and a second movable partition which section the respective chambers and change the volumes of the chambers; channels each of which penetrates the corresponding one of the first and second movable partitions so that the two adjoining chambers can communicated with each other; and non-return valves each of which opens and closes the corresponding one of the channels. Each of the non-return valves closes when the corresponding movable partition shifts from the inlet channel side toward the outlet channel side, and opens when the corresponding movable partition shifts from the outlet channel side toward the inlet channel side.

### BACKGROUND

1. Technical Field

The present invention relates to a liquid supply pump, and a medical instrument including this liquid supply pump.

2. Related Art

A liquid supply pump which includes a plurality of liquid pressurizing and supplying units disposed in series for pressurizing and supplying a small amount of liquid is known. According to this liquid supply pump, the plural liquid pressurizing and supplying units are sequentially operated in the order of the locations of the units from the upstream side to the downstream side, so that liquid can flow in accordance with suction and pressurized supply of liquid repeated by the liquid pressurizing and supplying units (for example, see JP-A-2008-2335).

According to the technology disclosed in JP-A-2008-2335, the liquid pressurizing and supplying unit disposed on the most upstream side sucks liquid and prevents reverse flow of liquid toward the upstream side. The liquid pressurizing and supplying unit disposed at the intermediate position pressurizes and supplies liquid toward the downstream side. The liquid pressurizing and supplying unit disposed on the most downstream side discharges liquid and prevents reverse flow of liquid toward the upstream side. According to this structure, there is a period when the liquid pressurizing and supplying unit disposed on the most downstream side closes its channel for preventing reverse flow of liquid. In this case, liquid flows as pulses instead of continuous streams, in which condition continuous supply of a small and constant amount of liquid becomes difficult.

### SUMMARY

An advantage of some aspects of the invention is to solve at least a part of the aforementioned problems and the invention can be implemented as the following forms or application examples.

### APPLICATION EXAMPLE 1

This application example of the invention is directed to a liquid supply pump including: three liquid chambers disposed in series and produced by dividing a space between an inlet channel into which liquid is supplied and an outlet channel from which liquid is delivered; two movable partitions which section the liquid chambers and change the volumes of the liquid chambers; a driving member which alternately reciprocates the two movable partitions between the inlet channel and the outlet channel; a motor which gives a driving force to the driving member; channels each of which penetrates the corresponding movable partition so that the two adjoining liquid chambers can communicated with each other; and non-return valves each of which opens and closes the corresponding channel. Each of the non-return valves closes the corresponding channel when the movable partition containing this channel shifts in such a direction as to decrease the volume of the corresponding liquid chamber, and opens the corresponding channel when the movable partition containing this channel shifts in such a direction as to increase the volume of the corresponding liquid chamber.

According to this application example of the invention, each of the non-return valves opens to introduce liquid into the liquid chamber when the movable partition shifts in such a direction as to increase the volume of the liquid chamber, and closes to supply liquid toward the outlet channel when the movable partition shifts in such a direction as to decrease the volume of the liquid chamber. According to this structure, a constant amount of liquid can be supplied in continuous streams by alternately shifting the two partitions forward and backward. Moreover, one of the non-return valves closes when the other non-return valve opens. Thus, reverse flow of liquid from the downstream side can be prevented.

### APPLICATION EXAMPLE 2

It is preferable that the liquid supply pump of the above application example is configured such that the two movable partitions change the volumes of the liquid chambers substantially at the same speed, and one of the two movable partitions shifts in such a direction as to decrease the volume of the corresponding liquid chamber when the other movable partition shifts in such a direction as to increase the volume of the corresponding liquid chamber.

According to this structure, the volume change speeds of the movable partitions are substantially uniform, and one of the movable partitions constantly shifts in such a direction as to decrease the volume of the liquid chamber. Thus, variations in the flow speed (flow amount) can be reduced, and the constant liquid supply amount can be maintained per unit period.

### APPLICATION EXAMPLE 3

It is preferable that the liquid supply pump of the above application example further includes a liquid supply unit including the liquid chambers and the movable partitions, and a driving unit including the driving member and the motor. In this case, the liquid supply unit and the driving unit are connected with each other in such a manner as to be detachable from each other.

There is a possibility of corrosion of the components included in the liquid supply unit in contact with liquid depending on the types of liquid to be supplied. According to this structure, the liquid supply unit and the driving unit are detachable from each other, and the liquid supply unit can be replaced with new one for avoiding operation failure or clogging of the channels due to corrosion of the components or the like. Moreover, repeated use of the driving unit can reduce the running cost.

### APPLICATION EXAMPLE 4

It is preferable that the liquid supply pump of the above application example further includes a liquid supply unit including the liquid chambers, the movable partitions, and the driving member, and a motor unit including the main body of the motor and a transmission gear train. In this case, the liquid supply unit and the motor unit are connected with each other in such a manner as to be detachable from each other.

According to this structure, the motor unit and the liquid supply unit are provided as separate units. In this case, the size limitation to the motor decreases, which allows the use of a high-output motor and thus realizes stabilized operation.

### APPLICATION EXAMPLE 5

It is preferable that the driving member of the liquid supply pump according to the above application example includes a cam mechanism which presses each of the movable partitions in such a direction as to decrease the volume of the corresponding liquid chamber, and an elastic member which pushes back each of the movable partitions in such a direction as to increase the volume of the corresponding liquid chamber.

According to the liquid supply pump disclosed in JP-A-2008-2335, each of the liquid pressurizing and supplying units includes a diaphragm and an electromagnetic solenoid having a permanent magnet. Thus, the structure of the liquid pressurizing and supplying units becomes complicated. Moreover, a control device is required for separately controlling each operation of the plural liquid pressurizing and supplying units. In this case, matching the timing for operating each of the liquid pressurizing and supplying units is difficult.

According to this application example of the invention, however, the cam mechanism is employed as the driving member. In this case, the timing for operating the movable partitions is determined based on the phase difference of the cams. Thus, appropriate timing can be determined by setting an accurate and arbitrary phase difference. Moreover, the movable partitions are pushed back by using the elastic member (such as a spring). Thus, simplification of the structure can be achieved. According to this structure, the liquid supply speed can be easily controlled by varying the rotation speed of the motor.

### APPLICATION EXAMPLE 6

This application example of the invention is directed to a medical instrument including: the liquid supply pump according to any one of the above application examples; a pulse generator which receives liquid from the liquid supply pump and converts the liquid into pulsed liquid; and a nozzle which ejects the pulsed liquid produced by the pulse generator as liquid drops in pulses.

The pulse generator can convert liquid into pulsed liquid and eject the pulsed liquid at high speed. Thus, the medical instrument can execute excision, incision, exfoliation or the like of tissue while preserving capillaries including blood vessels in a preferable condition. The pulse generator of this type needs to receive a constant flow amount of liquid for stabilized operation. According to this application example of the invention, the liquid supply pump of the above application example can continuously supply a constant flow amount of liquid to the pulse generator, and thus can achieve stabilized operation.

### BRIEF DESCRIPTION OF THE DRAWINGS

The invention will be described with reference to the accompanying drawings, wherein like numbers reference like elements.

FIG. 1 is a cross-sectional view of a liquid supply pump taken along a line extending in the liquid supply direction according to a first embodiment.

FIG. 2 illustrates the shapes and phases of a first cam, a second cam, and a third cam.

FIG. 3 shows the relationship between the rotation angle and the stroke of a cam mechanism.

FIG. 4 illustrates the relationship between cams and rods as viewed from the cam mechanism.

FIG. 5 is a front view illustrating a cam shaft as viewed in the axial direction.

FIGS. 6A through 6F illustrate liquid supply conditions of the liquid supply pump.

FIG. 7 is a chart showing the strokes of a first movable partition and a second movable partition.

FIG. 8 is a cross-sectional view illustrating apart of a liquid supply unit according to a modified example 1.

FIG. 9 is a cross-sectional view illustrating apart of a liquid supply unit according to a modified example 2.

FIG. 10 is a partial cross-sectional view illustrating the connection area between a liquid supply unit and a motor unit according to a second embodiment.

FIG. 11 illustrates the general structure of a medical instrument.

### DESCRIPTION OF EXEMPLARY EMBODIMENTS

Exemplary embodiments according to the invention are hereinafter described with reference to the drawings.

The figures referred to herein are schematic illustrations whose reduction scales for components and parts in the horizontal and vertical directions are different from the actual scales for easy understanding of the figures.

### First Embodiment

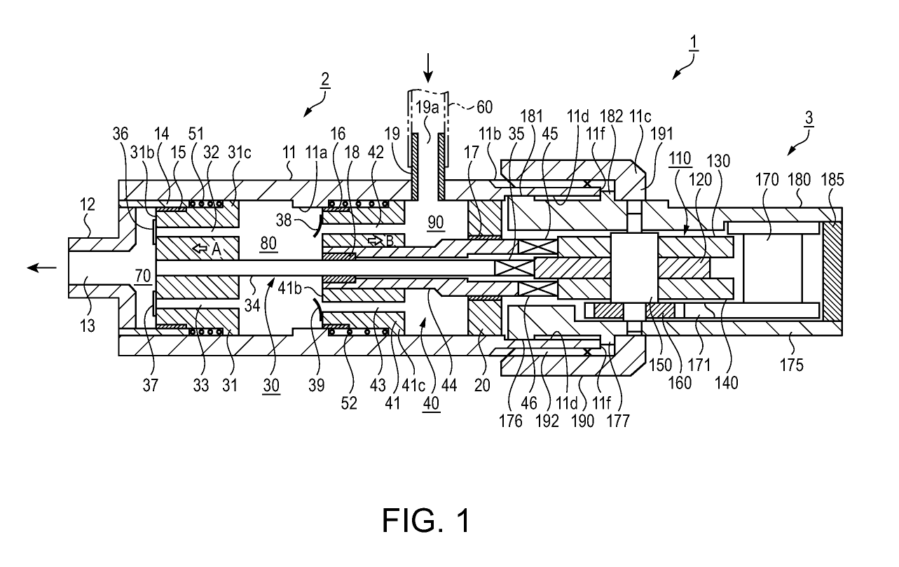

FIG. 1 is a cross-sectional view of a liquid supply pump taken along a line extending in the direction of liquid supply according to a first embodiment. As illustrated in FIG. 1, a liquid supply pump 1 includes a liquid supply unit 2 and a driving unit 3 detachably joined to each other by a connecting member 190.

The structure of the liquid supply unit 2 is initially explained.

The liquid supply unit 2 includes a first pressurizing and supplying unit 30 and a second pressurizing and supplying unit 40 within a cylindrical housing tube 11. The housing tube 11 has a liquid supply tube 19 which contains an inlet channel 19a opened on the side surface of the housing tube 11 in the vicinity of the driving unit 3, and an outlet tube 12 which contains an outlet channel 13 opened at the end of the housing tube 11 on the side opposite to the driving unit 3. The first pressurizing and supplying unit 30 and the second pressurizing and supplying unit 40 have a first movable partition 31 and a second movable partition 41, respectively. The first movable partition 31 and the second movable partition 41 divide the interior of the housing tube 11 between the inlet channel 19a and the outlet channel 13 into three parts of a first liquid chamber 70, a second liquid chamber 80, and a third liquid chamber 90 disposed in series. A tube 60 is connected with the liquid supply tube 19 so that liquid can be supplied from a not-shown liquid container through the tube 60 to the third liquid chamber 90 substantially at constant pressure.

The third liquid chamber 90 is formed by the inner circumferential surface of the housing tube 11, the second movable partition 41, and a seal ring 20. The seal ring 20 is closely fixed to the inner circumference of the housing tube 11. The inlet channel 19a communicates with the third liquid chamber 90. Thus, the third liquid chamber 90 functions as a liquid supply chamber.

The first pressurizing and supplying unit 30 has the first movable partition 31 and a rod 34 inserted through the center of the cross section of the first movable partition 31 by press fit. The first movable partition 31 is a disk-shaped component which has an outer circumference sliding on the inner circumferential surface of the housing tube 11. Channels 32 and 33 penetrate the first movable partition 31 so that the first liquid chamber 70 and the second liquid chamber 80 can communicate with each other through the channels 32 and 33. The number of these channels is not limited to two. Non-return valves 36 and 37 are fixed to an end surface 31b of the channels 32 and 33 in the vicinity of the first liquid chamber 70.

The rod 34 penetrates the second movable partition 41 and a rod 44 and extends toward the driving unit 3. A part of the side surface at the tip of the rod 34 is cut into a flat surface as a cam pressing portion 35 in contact with the cam surface of a first cam 120. FIG. 1 illustrates a condition in which the first cam 120 presses the first movable partition 31 via the rod 34 in such a direction as to decrease the volume of the first liquid chamber 70 (direction of arrow A). Under this condition, the non-return valves 36 and 37 close the channels 32 and 33 and reduce the volume of the first liquid chamber 70, thereby discharging liquid through the outlet channel 13.

When shifting toward the outlet channel 13, the first movable partition 31 deforms a coil spring 51 provided as an elastic member. The coil spring 51 is attached between a spring regulation pipe 14 and a spring pressing portion 31c formed on the first movable partition 31. The first movable partition 31 is pushed back toward the second movable partition 41 by the elastic force of the coil spring 51 when the first cam 120 releases its press against the rod 34. In this condition, the non-return valves 36 and 37 open the channels 32 and 33, and liquid flows into the first liquid chamber 70. Thus, the coil spring 51 functions as a driving member in cooperation with a cam mechanism 110.

The second movable partition 41 has a sealing member 18 which prevents leakage of liquid between the rod 34 and the second movable partition 41. Moreover, a sealing member 15 is also equipped between the sliding surfaces of the first movable partition 31 and the housing tube 11 (on the inner circumferential surface of the spring regulation pipe 14 in the figure) to prevent leakage of liquid between the first liquid chamber 70 and the second liquid chamber 80.

A not-shown rotation stopper for the first movable partition 31 is provided between the outer circumference of the first movable partition 31 and the inner circumference of the housing tube 11.

The number of the non-return valves 36 and 37 may be determined in accordance with the number of the channels. Alternatively, a one-piece member which has the same number of valve bodies as the number of the channels as parts capable of opening and closing the channels may be provided as the non-return valves 36 and 37.

The second pressurizing and supplying unit 40 has the second movable partition 41 and the rod 44 inserted through the center of the cross section of the second movable partition by press fit. The second movable partition 41 is a disk-shaped component which has an outer circumference sliding on the inner circumferential surface of the housing tube 11. Channels 42 and 43 penetrate the second movable partition 41 so that the second liquid chamber 80 and the third liquid chamber 90 can communicate with each other through the channels 42 and 43. The number of these channels is not limited to two similarly to the first movable partition 31. Non-return valves 38 and 39 are fixed to an end surface 41b of the channels 42 and 43 in the vicinity of the second liquid chamber 80.

The rod 44 penetrates the seal ring 20 and extends toward the driving unit 3. The tip of the rod 44 is divided into two parts the side surfaces of which are partially cut into flat surfaces as cam pressing portions 45 and 46, respectively. The cam pressing portion 45 contacts the cam surface of a second cam 130, while the cam pressing portion 46 contacts the cam surface of a third cam 140. The cam pressing portions 45 and 46 are symmetric with respect to the center axis of the rod 44. The second cam 130 and the third cam 140 have the same shape and the same phase difference from the first cam 120. Thus, necessary functions can be provided even when only either the cam pressing portion 45 or the cam pressing portion 46, and either the second cam 130 or the third cam 140 are equipped.

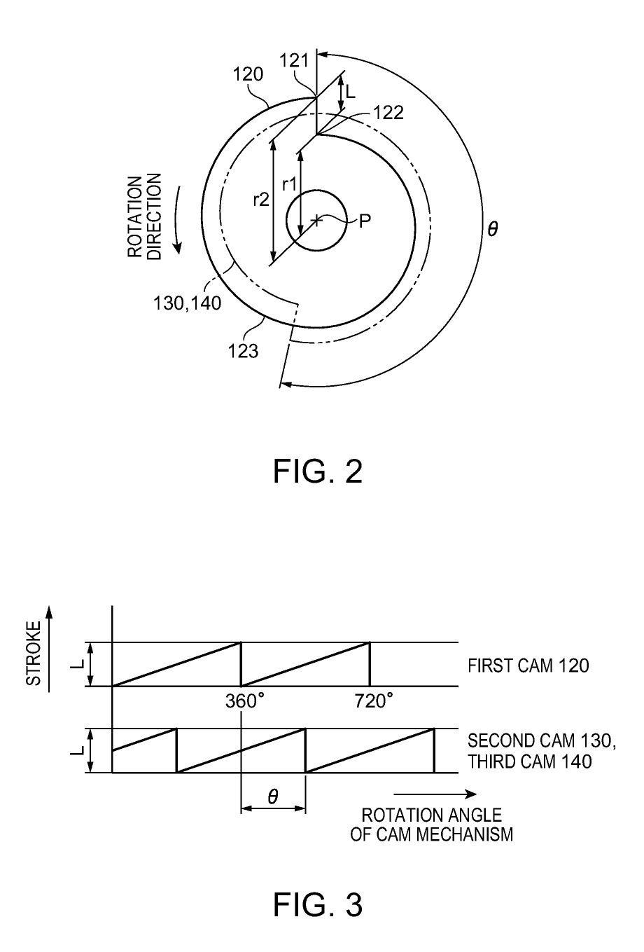

FIG. 1 illustrates a condition in which the second movable partition 41 shifts in such a direction as to decrease the volume of the third liquid chamber 90 (direction of arrow B). Under this condition, the non-return valves 38 and 39 open the channels 42 and 43 and reduce the volume of the third liquid chamber 90, whereby liquid flows from the third liquid chamber 90 to the second liquid chamber 80. When the engagement between the rod 44 and the top portions of the second and third cams 130 and 140 (see FIG. 2) is released, the second movable partition 41 is pushed back in such a direction as to decrease the volume of the third liquid chamber 90 by the elastic force of a coil spring 52 as an elastic member. Thus, the coil spring 52 functions as a driving member in cooperation with the cam mechanism 110.

The coil spring 52 is attached between a spring regulation portion 11a formed on the inner circumferential surface of the housing tube 11 and a spring pressing portion 41c formed on the outer circumference of the second movable partition 41. When the second movable partition 41 is pressed by the second cam 130 and the third cam 140 to shift in such a direction as to decrease the volume of the second liquid chamber 80, the non-return valves 38 and 39 close the channels 42 and 43 along with deformation of the coil spring 52.

The seal ring 20 has a sealing member 17 which prevents leakage of liquid between the rod 44 and the seal ring 20. Moreover, a sealing member 16 is provided between the sliding surfaces of the second movable partition 41 and the housing tube 11 to prevent leakage of liquid between the second liquid chamber 80 and the third liquid chamber 90.

A not-shown rotation stopper for the second movable partition 41 is provided between the outer circumference of the second movable partition 41 and the inner circumference of the housing tube 11.

The number of the non-return valves 38 and 39 may be determined in accordance with the number of the channels. Alternatively, a one-piece member which has the same number of valve bodies as the number of the channels as parts capable of opening and closing the channels may be provided as the non-return valves 38 and 39.

The structure of the driving unit 3 is now explained with reference to FIG. 1. The driving unit 3 has the cam mechanism 110 and a motor 170 as the driving member held between a first device frame 175 and a second device frame 180.

The cam mechanism 110 has a cam shaft 150, and the first cam 120, the second cam 130, the third cam 140, and a cam gear 160 engaging with the cam shaft 150. The respective shapes of the first cam 120, the second cam 130, and the third cam 140 will be described later with reference to FIGS. 2 and 3. The cam mechanism 110 is supported between the first device frame 175 and the second device frame 180 in such a manner as to be rotatable. The first device frame 175 and the second device frame 180 are fixed to each other by screws or the like with a third device frame 185 provided between the first and second device frames 175 and 180. The first device frame 175 and the second device frame 180 are so shaped as to close the periphery of the first cam 120, the second cam 130, and the third cam 140 except for the junction area between the liquid supply unit 2 and the driving unit 3 through which a part of the first, second, and third cams 120, 130, and 140 is exposed.

The motor 170 has a motor main body and a transmission gear train 171, and is fixed to the second device frame 180. The end gear of the transmission gear train 171 engages with the cam gear 160 to transmit the rotation of the motor 170 to the cam mechanism 110.

The driving unit 3 is molded in such a shape that its junction area connected with the liquid supply unit 2 can engage with the inner circumference of the housing tube 11 after assembly of the cam mechanism 110 and the motor 170. A positioning groove 11d extending in the axial direction is formed on the inner circumferential surface of the housing tube 11. Projections 176 and 181 provided on the first device frame 175 and the second device frame 180 of the driving unit 3, respectively, slide along the positioning groove 11d to be attached thereto to prevent rotation of the first and second device frame 175 and 180 relative to the housing tube 11.

The cross-sectional areas of the first liquid chamber 70 and the second liquid chamber 80 in the direction perpendicular to the shift directions of the respective movable partitions 31 and 41 are equalized with each other. Since the second liquid chamber 80 contains the rod 34, the cross-sectional area of the first liquid chamber 70 is decreased by the area corresponding to the rod 34. This structure is required so as to keep the liquid supply amount substantially constant during uniform movement of the first movable partition 31 and the second movable partition 41.

The junction structure of the liquid supply unit 2 and the driving unit 3 is now explained with reference to FIG. 1.

A screw (male screw) 11b is provided at the end of the housing tube 11 in the vicinity of the driving unit 3. Fixing projections 177 and 182 are provided at the end of the outer circumferential surface of the driving unit 3 in the vicinity of the liquid supply unit 2.

The connecting member 190 has a cylindrical shape, and includes a pressing portion 191 projecting toward the inner circumference, and a screw 192. For assembly, the connecting member 190 is inserted from the end of the driving unit 3 (right side in the figure), and the screw 11b and the screw 192 are brought into screw-engagement with each other, in which condition the fixing projections 177 and 182 are pressed against a receiving portion 11f by the connecting member 190 for junction between the liquid supply unit 2 and the driving unit 3. The liquid supply unit 2 and the driving unit 3 joined to each other by this method can be separated from each other by release of the screw-engagement of the connecting member 190.

The cam mechanism 110 is now explained with reference to the drawings.

FIG. 2 illustrates the shapes and the phases of the first cam, the second cam, and the third cam. FIG. 3 shows the relationship between the rotation angle and the stroke of the cam mechanism. The upper part in FIG. 3 corresponds to the first cam 120. The lower part in FIG. 3 corresponds to the second cam 130 and the third cam 140. In this embodiment, the shapes of the first cam 120, the second cam 130, and the third cam 140 are uniform. The cam shape of the first cam 120 is herein explained as a typical example.

The first cam 120 has the cam surface constituted by the outer circumferential surface. The cam surface of the first cam 120 has a spiral shape extending from a bottom portion 122 to a top portion 121, and has a uniform distance from a rotation center P per unit rotation angle. It is assumed that the distance between the rotation center P and the bottom portion 122 and the distance between the rotation center P and the top portion 121 are r1 and r2, respectively. As can be seen from FIG. 1, the rod 34 is urged toward the first cam 120 by the coil spring 51. Thus, the first movable partition 31 increases the volume of the first liquid chamber 70 to the maximum when the tip of the cam pressing portion 35 of the rod 34 contacts the bottom portion 122.

In accordance with rotation of the first cam 120, a pressing surface 123 shifts the first movable partition 31 in such a direction as to decrease the volume of the first liquid chamber 70. When the top portion 121 reaches the cam pressing portion 35, the volume of the first liquid chamber 70 becomes the minimum. With further rotation of the first cam 120, the engagement between the rod 34 and the top portion 121 is released, whereupon the cam pressing portion 35 of the rod 34 is pushed back by the elastic force of the coil spring 51 until the cam pressing portion 35 contacts the bottom portion 122.

The second cam 130 and the third cam 140 have shapes similar to the shape of the first cam 120, and perform operations for shifting the second movable partition 41 in a manner similar to the corresponding operation of the first cam 120. However, the second cam 130 and the third cam 140 have a phase difference of an angle θ from the first cam 120. The first cam 120, the second cam 130, and the third cam 140 rotate at the same speed.

Thus, as illustrated in FIG. 3, the change of the stroke (inclination) of each cam with respect to the rotation angle is the same, but includes the phase difference of the angle θ. In this case, either the first cam 120 or the second cam 130 (third cam 140) constantly shifts the corresponding movable partition so that liquid can be kept delivered.

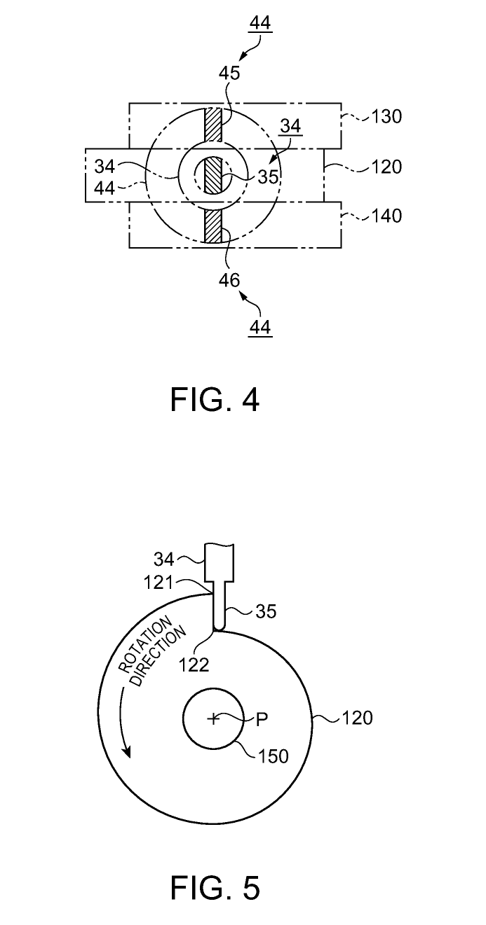

For appropriate contact between the cam pressing portion of each rod and the cam surface of the corresponding cam, a part of the side surface at the tip of the cam pressing portion is cut into a flat surface. The details of this structure are now explained with reference to FIGS. 4 and 5.

FIG. 4 illustrates the relationship between the cam and the rod as viewed from the cam mechanism. FIG. 5 is a front view of the cam shaft as viewed in the axial direction. FIG. 5 shows the rod 34 and the first cam 120 as an example. As illustrated in FIGS. 4 and 5, a part of the side surface at each tip of the cam pressing portion 35 of the rod 34 and the cam pressing portions 45 and 46 of the rod 44 is cut into a flat surface. Thus, the cam pressing portion 35 having passed over the top portion 121 in accordance with rotation of the first cam 120 is pushed back toward a position close to the bottom portion 122. According to this structure, the cam pressing portion 35 shifts the first movable partition 31 forward and backward in accordance with the rotation of the first cam 120 in the manner illustrated in the chart in FIG. 3 showing the relationship between the stroke and the rotation angle.

The operation for liquid supply according to this embodiment is now explained with reference to the drawings.

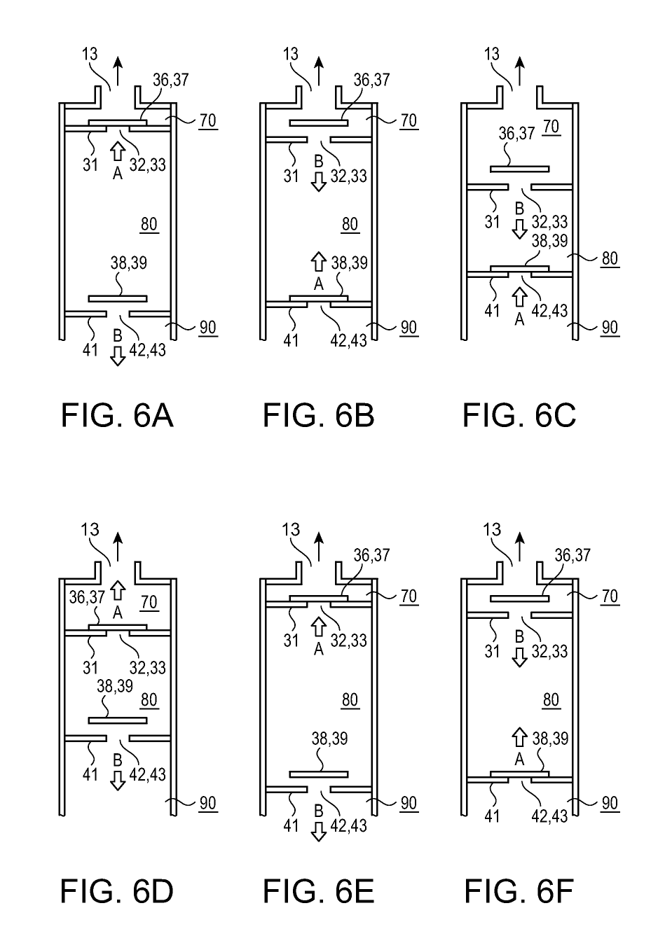

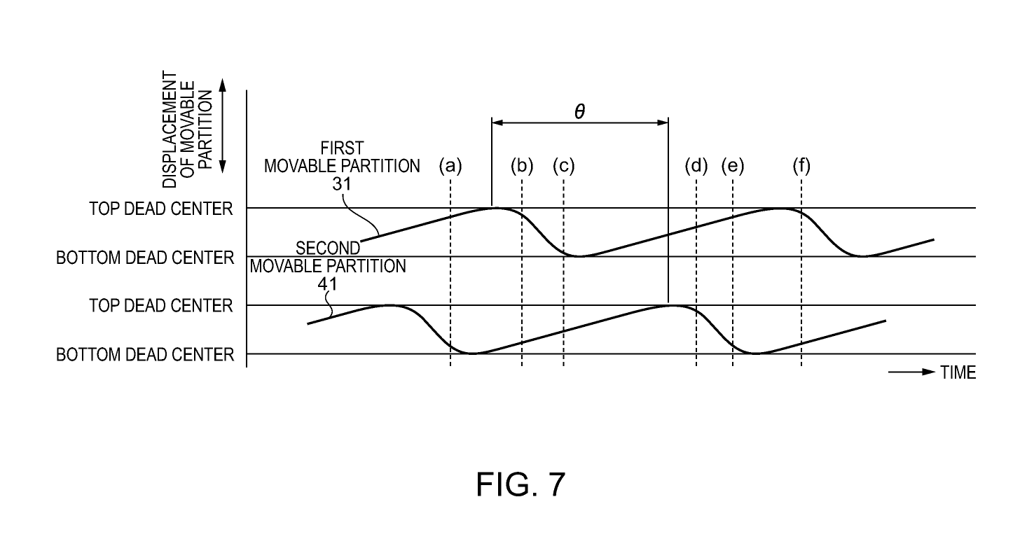

FIGS. 6A through 6F illustrate liquid supply conditions of the liquid supply pump. FIG. 7 is a chart showing the strokes of the first movable partition and the second movable partition. FIGS. 6A through 6F show the simplified structure. In FIG. 7, the horizontal axis indicates the elapsed time, and the vertical axis indicates the shift strokes of the first movable partition 31 and the second movable partition 41. The top dead center corresponds to the positions of the respective movable partitions shifted closest to the outlet channel. The bottom dead center corresponds to the positions of the respective movable partitions shifted closest to the inlet channel. The operation is herein explained by contrast between FIGS. 6A through 6F and FIG. 7.

FIG. 6A shows a condition in which the first movable partition 31 is positioned before the top dead center. In this case, the second movable partition 41 is positioned immediately before the bottom dead center. In this condition, the non-return valves 36 and 37 close the channels 32 and 33 and keep reducing the volume of the first liquid chamber 70 to discharge the liquid within the first liquid chamber 70 through the outlet channel 13. Thus, the amount of liquid corresponding to the reduction of the volume of the first liquid chamber 70 flows out. Under the condition in which the second movable partition 41 is positioned immediately before the bottom dead center, the non-return valves 38 and 39 open the channels 42 and 43. In this case, liquid flows from the third liquid chamber 90 toward the second liquid chamber 80. Thus, the amount of liquid corresponding to the reduction of the volume of the third liquid chamber 90 flows into the second liquid chamber 80. This condition is indicated at a position (a) in FIG. 7.

FIG. 6B shows a condition in which the first movable partition 31 is positioned immediately after the upper dead center. In this case, the second movable partition 41 passes through the bottom dead center and starts reducing the volume of the second liquid chamber 80. In this condition, the non-return valves 36 and 37 open the channels 32 and 33, while the non-return valves 38 and 39 close the channels 42 and 43. Thus, the liquid within the second liquid chamber 80 passes through the channels 32 and 33 and the first liquid chamber 70, and flows out through the outlet channel 13. At this time, liquid at the inlet channel 19a is sucked in accordance with the gradual increase in the volume of the third liquid chamber 90, and flows into the third liquid chamber 90. This condition is indicated at a position (b) in FIG. 7.

The stroke of the first movable partition 31 at the top dead center is designed to change its inclination direction linearly between the positive direction and the negative direction as illustrated in FIG. 3. In practice, however, this stroke gradually changes by the effect of the inertia of the first movable partition 31, the fluid resistance of the liquid, the internal pressure, or the elastic force of the coil spring 51.

FIG. 6C illustrates a condition in which the first movable partition 31 and the second movable partition 41 are further shifted from the positions shown in FIG. 6B. This condition is indicated at a position (c) in FIG. 7. As illustrated in the figure, liquid is kept delivered from the outlet channel 13 by the shift of the second movable partition 41 similarly to the condition shown in FIG. 6B. Thus, liquid flows from the second liquid chamber 80 into the first liquid chamber 70.

FIG. 6D illustrates a condition in which the first movable partition 31 and the second movable partition 41 are further shifted from the positions shown in FIG. 6C. This condition is indicated at a position (d) in FIG. 7. As illustrated in the figure, the first movable partition 31 passes through the bottom dead center, whereat the non-return valves 36 and 37 close the channels 32 and 33. On the other hand, the second movable partition 41 passes through the top dead center, whereat the non-return valves 38 and 39 open the channels 42 and 43. In this condition, liquid is delivered from the outlet channel 13 by the shift of the first movable partition 31 . Thus, liquid flows from the third liquid chamber 90 into the second liquid chamber 80.

FIG. 6E illustrates a condition in which the first movable partition 31 and the second movable partition 41 are further shifted from the positions shown in FIG. 6D. This condition is indicated at a position (e) in FIG. 7. As illustrated in the figure, liquid is kept delivered from the outlet channel 13 by the shift of the first movable partition 31 similarly to the condition shown in FIG. 6A. Thus, liquid flows from the third liquid chamber 90 into the second liquid chamber 80.

FIG. 6F illustrates a condition in which the first movable partition 31 and the second movable partition 41 are further shifted from the positions shown in FIG. 6E. This condition is indicated at a position (f) in FIG. 7. As illustrated in the figure, the first movable partition 31 passes through the top dead center, whereat the non-return valves 36 and 37 open the channels 32 and 33. On the other hand, the second movable partition 41 passes through the bottom dead center, whereat the non-return valves 38 and 39 close the channels 42 and 43. Thus, liquid flows out from the outlet channel 13 by the shift of the second movable partition 41 similarly to the condition shown in FIG. 6B.

As illustrated in FIG. 7, the shift range of the first movable partition 31 from the top dead center to the bottom dead center corresponds to the opening range of the non-return valves 36 and 37, and the shift range of the first movable partition 31 from the bottom dead center to the top dead center corresponds to the closing range of the non-return valves 36 and 37. On the other hand, the shift range of the second movable partition 41 from the top dead center to the bottom dead center corresponds to the opening range of the non-return valves 38 and 39, and the shift range of the second movable partition from the bottom dead center to the top dead center corresponds to the closing range of the non-return valves 38 and 39. The angle θ between the top dead center of the first movable partition 31 and the top dead center of the second movable partition 41 corresponds to the phase difference between the top position of the first cam 120 and the top positions of the second cam 130 and the third cam 140.

According to this structure, the non-return valves provided on either the first movable partition 31 or the second movable partition 41 close the corresponding channels, and simultaneously the non-return valves provided on the other movable partition 31 or 41 open the corresponding channels, which condition is constantly produced as illustrated in FIG. 7. Since the inclination of each stroke of the first movable partition 31 and the second movable partition 41 is equalized, a constant flow amount can be continuously delivered during operation of the liquid supply pump 1.

According to the first embodiment, therefore, the first movable partition 31 shifting from the outlet channel 13 side toward the inlet channel 19a side expands the volume of the first liquid chamber 70 while opening the non-return valves 36 and 37 so that liquid can flow into the first liquid chamber 70. The first movable partition 31 shifting from the inlet channel 19a side toward the outlet channel 13 side reduces the volume of the first liquid chamber 70 while closing the non-return valves 36 and 37 so that liquid can flow toward the outlet channel 13. On the other hand, the second movable partition 41 shifting from the outlet channel 13 side toward the inlet channel 19a side expands the volume of the second liquid chamber 80 while opening the non-return valves 38 and 39 so that liquid can flow into the second liquid chamber 80. The second movable partition 41 shifting from the inlet channel 19a side toward the outlet channel 13 side reduces the volume of the second liquid chamber 80 while closing the non-return valves 38 and 39 so that liquid can pass through the first liquid chamber 70 and flow out through the outlet channel 13.

By alternate movement of the two movable partitions 31 and 41 forward and backward, a constant amount of liquid can be delivered as continuous streams. In this structure, one of the non-return valves closes when the other non-return valve opens, which prevents reverse flow of liquid from the downstream side.

The first cam 120 and the second and third cams 130 and 140 shifting the two movable partitions have the same shape and rotate around the same axis. In this case, the volume change speeds of the first liquid chamber 70 and the second liquid chamber 80 become substantially equivalent, and either one of the two movable partitions 31 and 41 constantly shifts in such a direction as to decrease the volume of the first liquid chamber 70 or the second liquid chamber 80. Both the substantially uniform volume change speed and the constant shift of one of the movable partitions 31 and 41 toward the outlet channel 13 can reduce variations in the flow amount and keep the constant liquid supply amount per unit period.

The liquid supply unit 2 and the driving unit 3 are detachably joined to each other by the connecting member 190. There is a possibility of corrosion of the components included in the liquid supply unit 2 in contact with liquid depending on the types of liquid to be supplied. According to this structure, the liquid supply unit 2 detachable from the driving unit 3 can be replaced with new one for avoiding operation failure or clogging of the channels. On the other hand, the driving unit 3 not directly contacting liquid can be repeatedly used, which reduces the running cost.

The driving member has the cam mechanism 110, and the coil springs 51 and 52. According to this structure, the timing for shifting the first movable partition 31 and the second movable partition 41 is determined based on the phase difference between the first cam 120 and the second and third cams 130 and 140. In this case, an accurate and arbitrary phase difference can be set for determining appropriate timing. Moreover, the first movable partition 31 and the second movable partition 41 are pushed back by the coil springs 51 and 52, which contributes to simplification of the structure. This structure also allows easy control of the liquid supply speed which only requires change of the rotation speed of the motor 170.

The liquid supply pump 1 thus constructed can be modified in the following manners.

### MODIFIED EXAMPLE 1

A modified example 1 is now described with reference to the associated drawing. In the modified example 1, the coil springs 51 and 52 for pushing back the first movable partition 31 and the second movable partition 41 are disposed within the first liquid chamber 70 and the second liquid chamber 80. The components and parts in this example corresponding to the same components and parts in the first embodiment (see FIG. 1) are given the same reference numbers. The differences in this example from the first embodiment are chiefly explained herein, showing the first pressurizing and supplying unit 30 as an example.

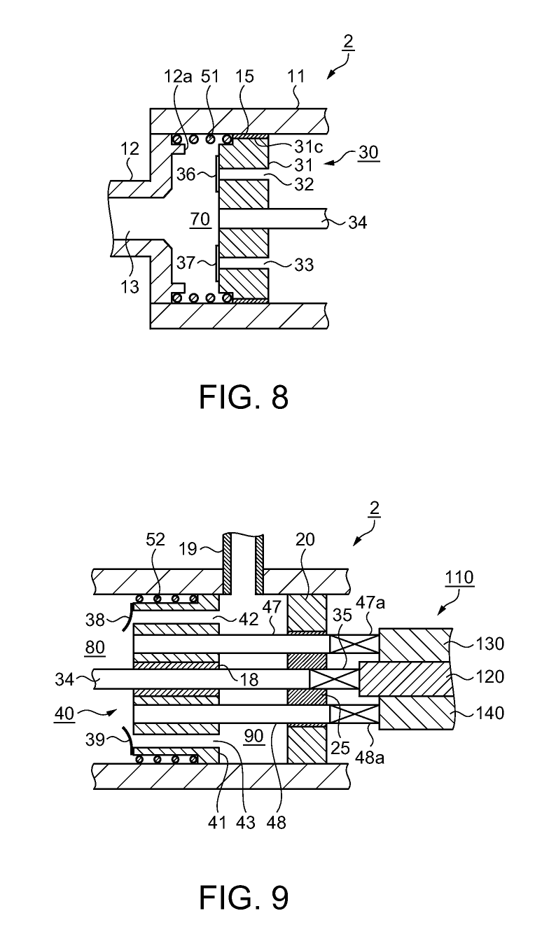

FIG. 8 is a cross-sectional view illustrating apart of a liquid supply unit according to the modified example 1. As illustrated in FIG. 8, the first pressurizing and supplying unit 30 is disposed within the housing tube 11. The first pressurizing and supplying unit 30 includes the first movable partition 31 which has the non-return valves 36 and 37 for opening and closing the channels 32 and 33, and the rod 34 which shifts the first movable partition 31 toward the outlet channel 13. The sealing member 15 is provided between the first movable partition 31 and the inner circumference of the housing tube 11. A step 31c is formed on the outer circumferential surface of the first movable partition 31.

A ring-shaped spring regulation portion 12a projecting toward the inside of the first liquid chamber 70 is formed on the outlet tube 12 to which the outlet channel 13 is opened. The coil spring 51 which pushes back the first pressurizing and supplying unit 30 in such a direction as to increase the volume of the first liquid chamber 70 is disposed within the first liquid chamber 70 between the first movable partition 31 and the outlet tube 12. Thus, the position of the coil spring 51 is regulated between the step 31c of the first movable partition 31 and the spring regulation portion 12a of the outlet tube 12.

The relationship between the second movable partition 41, the spring regulation portion 11a, and the coil spring 52 included in the second pressurizing and supplying unit 40 (see FIG. 1) is similar to the corresponding relationship of the first pressurizing and supplying unit 30. Thus, the structure of the second pressurizing and supplying unit 40 is not specifically described nor depicted.

The liquid delivery operation according to this modified example is similar to the corresponding operation in the first embodiment. However, the number of the wires and the degree of freedom in designing the wire diameter of the coil spring 51 (and the coil spring 52) can be increased. Moreover, the thickness of the first movable partition 31 (and the second movable partition 41) in the forward and backward direction can be decreased, which reduces the size of the liquid supply unit 2.

### MODIFIED EXAMPLE 2

A modified example 2 is now described with reference to the associated drawing. In the modified example 2, two rods are provided to shift the second movable partition 41. The components and parts in this example corresponding to the same components and parts in the first embodiment (see FIG. 1) are given the same reference numbers. The differences in this example from the first embodiment are chiefly explained herein.

FIG. 9 is a cross-sectional view illustrating apart of a liquid supply unit according to the modified example 2. As illustrated in FIG. 9, the second pressurizing and supplying unit 40 is disposed within the housing tube 11. The second pressurizing and supplying unit 40 includes the second movable partition 41 which has the non-return valves 38 and 39 for opening and closing the channels 42 and 43, and rods 47 and 48 which shift the second movable partition 41 toward the second liquid chamber 80. The rods 47 and 48 are disposed substantially symmetric with respect to the rod 34 attached to the first movable partition 31.

The sealing member 18 is provided on the second movable partition 41 to seal the space between the rod 34 and the second movable partition 41 in such a manner as to slide along the space. A sealing member 25 is further provided on the seal ring 20 to seal the rods 34, 47 and 48 in such a manner as to slide along the rods 34, 47 and 48.

A part of each side surface at the tips of the rods 34, 47 and 48 contacting the first cam 120, the second cam 130, and the third cam 140, respectively, is cut into a flat surface as the cam pressing portion 35 and cam pressing portions 47a and 48a, respectively. Each shape of the cam pressing portions 35, 47a and 48a is the same as the corresponding shape in the first embodiment (see FIGS. 4 and 5).

According to this example, advantages similar to those in the first embodiment can be offered. Moreover, the shapes of the rods 47 and 48 shifting the second movable partition 41 can be simplified.

Such a structure which includes only a combination of either the rod 47 or 48 and either the second cam 130 or the third cam 140 for shifting the rod 47 or 48 is allowed. The structure shown in the modified example 1 may be incorporated in this example.

### Second Embodiment

A second embodiment is herein described with reference to the associated drawing. While the liquid supply pump 1 in the first embodiment (see FIG. 1) combines the liquid supply unit 2 and the driving unit 3 in such a manner as to be detachable from each other, in the second embodiment, a liquid supply unit and a motor unit are combined in such a manner as to be detachable from each other. The differences in this embodiment from the first embodiment are chiefly explained herein.

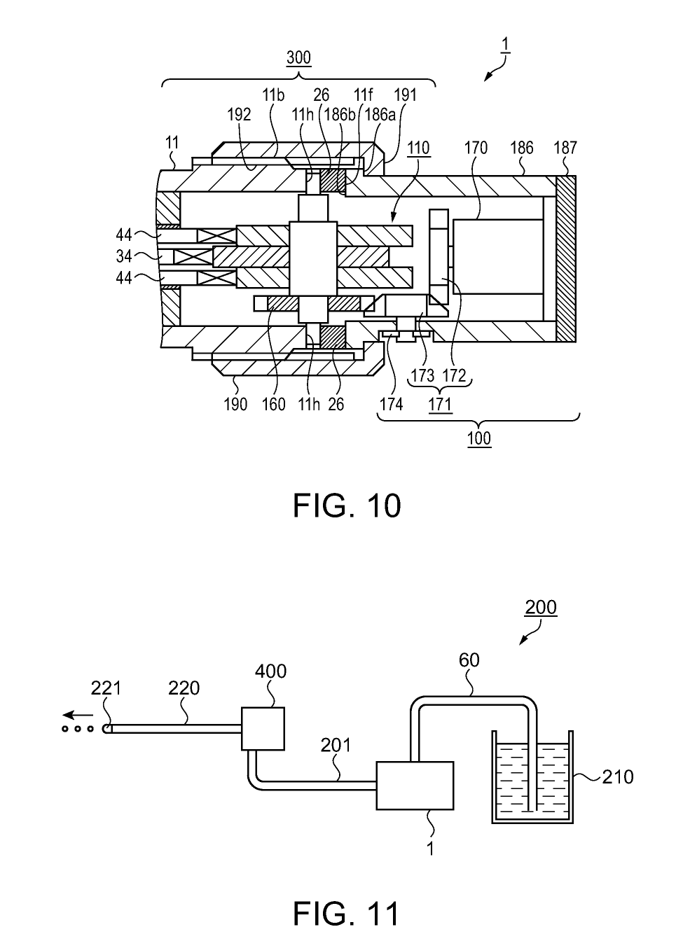

FIG. 10 is a partial cross-sectional view illustrating the junction area between the liquid supply unit and the motor unit according to the second embodiment. As illustrated in FIG. 10, the liquid supply pump 1 includes a liquid supply unit 300 and a motor unit 100 combined by the connecting member 190 in such a manner as to be detachable from each other.

The liquid supply unit 300 has the first pressurizing and supplying unit 30, the second pressuring and supplying unit 40 (see FIG. 1), and the cam mechanism 110 disposed within the housing tube 11. The first pressurizing and supplying unit 30, the second pressurizing and supplying unit 40, and the cam mechanism 110 have structures same as the corresponding structures in the first embodiment. A screw 11b (male screw) is formed at the end of the outer circumference of the housing tube 11 in the vicinity of the motor unit 100.

The cam mechanism 110 is supported by the cylindrical housing tube 11. The housing tube 11 has a slit-shaped cam mechanism support hole 11h opened from the end of the housing tube 11. For assembly, the cam mechanism 110 is inserted into the cam mechanism support hole 11h from the end of the housing tube 11, and a cam support member 26 is fitted to the cam mechanism support hole 11h to support the cam mechanism 110.

The motor unit 100 includes the motor 170 having a first transmission gear 172, and the transmission gear train 171, both disposed within a cylindrical motor frame 186. The transmission gear train 171 has the first transmission gear 172, and a second transmission gear 173 engaging with the first transmission gear 172. The second transmission gear 173 engaging with the first transmission gear 172 is a bevel gear disposed perpendicularly to the first transmission gear 172 to transmit the rotation of the motor 170. The second transmission gear 173 is supported on the outer circumference of the housing tube 11 by an E-ring 174 (or C-ring) . The end of the motor frame 186 is sealed by a bottom plate 187.

A ring-shaped fixing projection 186a which projects toward the outer circumference is provided at the end of the motor frame 186 in the vicinity of the liquid supply unit 300.

The connecting member 190 is a cylindrical component which includes the screw 192 (female screw) formed on the inner circumferential surface of the connecting member 190 and the ring-shaped pressing portion 191 projecting toward the inside. The connecting member 190 and the motor frame 186 engage with each other with play provided therebetween.

The liquid supply unit 300 and the motor unit 100 are connected with each other by engagement between the screw 192 of the connecting member 190 and the screw 11b of the housing tube 11. In this case, the receiving portion 11f of the housing tube 11 is brought into press contact with an end surface 186b of the motor frame 186 by the pressing portion of the connecting member 190. In the connection step, the cam gear 160 and the second transmission gear 173 are also brought into engagement with each other so that the rotation of the motor 170 can be transmitted to the cam mechanism 110.

According to this structure, the liquid supply unit 300 and the motor unit 100 are detachably attached to each other. In this case, the size limitation to the motor unit 100 decreases. Thus, the output of the motor 170 can be increased to a level sufficient for stabilizing its operation for the loads of the shifts of the cam mechanism 110, the first movable partition 31, and the second movable partition 41. Moreover, limitation to the number of the wires and the wire diameter of the coil springs 51 and 52 can be decreased. Thus, the forces required for pressing the coil springs 51 and 52 for deformation can be easily balanced against the forces of the coil springs 51 and 52 for pushing back the first movable partition 31 and the second movable partition 41.

### Medical Instrument

A medical instrument including the liquid supply pump 1 according to the respective embodiments and modified examples is herein explained.

FIG. 11 illustrates the general structure of the medical instrument. As illustrated in FIG. 11, a medical instrument 200 includes the liquid supply pump 1, and a pulse generator 400 which converts liquid supplied from the liquid supply pump 1 into pulsed liquid.

The liquid supply pump 1 connected with a liquid container 210 via the tube 60 (see FIG. 1) sucks liquid, and continuously supplies a constant amount of the liquid through a liquid supply tube 201 toward the pulse generator 400 at constant pressure.

The pulse generator 400 converts the supplied liquid into pulsed liquid, and ejects the pulsed liquid in the form of liquid drops through a delivery tube 220 and a nozzle 221 at high speed. The pulse generator 400 may have structure same as that of a pulse generator included in a fluid ejection device disclosed in JP-A-2008-82202.

According to this structure, the pulse generator 400 can convert liquid into pulsed liquid and eject the pulsed liquid in the form of liquid drops from the nozzle 221 at high speed. Thus, the medical instrument in this example can execute excision, incision, exfoliation or the like of tissue while preserving capillaries including blood vessels in a preferable condition. The pulse generator 400 of this type needs to receive liquid at a constant flow speed (flow amount) for stabilized operation. According to this example, the pulse generator 400 which uses the liquid supply pump 1 described in the respective embodiments and examples can achieve stabilized operation.

This application claims priority to Japanese Patent Application No. 2010-232255, filed on Oct. 15, 2010, the entirety of which is hereby incorporated by reference.

What is claimed is:

1. A liquid supply pump, comprising:
three chambers disposed in series and produced by dividing a space between an inlet channel into which liquid is supplied and an outlet channel from which liquid is delivered;
two movable partitions which section the chambers and change the volumes of the liquid chambers;
a driving member which alternately reciprocates the two movable partitions between the inlet channel and the outlet channel;
a motor which gives a driving force to the driving member;
channels each of which penetrates the corresponding movable partition so that the two adjoining chambers can communicated with each other; and
non-return valves each of which opens and closes the corresponding channel,
wherein each of the non-return valves closes the corresponding channel when the movable partition containing this channel shifts in such a direction as to decrease the volume of the corresponding liquid chamber, and opens the corresponding channel when the movable partition containing this channel shifts in such a direction as to increase the volume of the corresponding liquid chamber.

2. The liquid supply pump according to claim 1, wherein:
the two movable partitions change the volumes of the liquid chambers substantially at the same speed; and
one of the two movable partitions shifts in such a direction as to decrease the volume of the corresponding liquid chamber when the other movable partition shifts in such a direction as to increase the volume of the corresponding liquid chamber.

3. The liquid supply pump according to claim 1, further comprising:
a liquid supply unit including the liquid chambers and the movable partitions; and
a driving unit including the driving member and the motor,
wherein the liquid supply unit and the driving unit are connected with each other in such a manner as to be detachable from each other.

4. The liquid supply pump according to claim 1, further comprising:
a liquid supply unit including the liquid chambers, the movable partitions, and the driving member; and
a motor unit including the main body of the motor and a transmission gear train,
wherein the liquid supply unit and the motor unit are connected with each other in such a manner as to be detachable from each other.

5. The liquid supply pump according to claim 1, wherein the driving member includes a cam mechanism which presses each of the movable partitions in such a direction as to decrease the volume of the corresponding liquid chamber, and an elastic member which pushes back each of the movable partitions in such a direction as to increase the volume of the corresponding liquid chamber.

6. A medical instrument comprising:
the liquid supply pump according to claim 1;
a pulse generator which receives liquid from the liquid supply pump and converts the liquid into pulsed liquid; and
a nozzle which ejects the pulsed liquid produced by the pulse generator as liquid drops in pulses.

7. A medical instrument comprising:
the liquid supply pump according to claim 2;
a pulse generator which receives liquid from the liquid supply pump and converts the liquid into pulsed liquid; and
a nozzle which ejects the pulsed liquid produced by the pulse generator as liquid drops in pulses.

8. A medical instrument comprising:
the liquid supply pump according to claim 3;
a pulse generator which receives liquid from the liquid supply pump and converts the liquid into pulsed liquid; and
a nozzle which ejects the pulsed liquid produced by the pulse generator as liquid drops in pulses.

9. A medical instrument comprising:
the liquid supply pump according to claim 4;
a pulse generator which receives liquid from the liquid supply pump and converts the liquid into pulsed liquid; and
a nozzle which ejects the pulsed liquid produced by the pulse generator as liquid drops in pulses.

10. A medical instrument comprising:
the liquid supply pump according to claim 5;
a pulse generator which receives liquid from the liquid supply pump and converts the liquid into pulsed liquid; and
a nozzle which ejects the pulsed liquid produced by the pulse generator as liquid drops in pulses.
